(19) United States
(12) Patent Application Publication
(10) Pub. No.: US 2012/0210452 A1
(43) Pub. Date: Aug. 16, 2012

(54) ALKOXYLATED NON-IONIC ALKANOL ADJUVANT FORMULATIONS AND METHODS FOR MAKING AND USING THE SAME

(75) Inventors: Jeffrey Todd Langley, Rock Hill, SC (US); George Hudson, Rock Hill, SC (US)

(73) Assignee: FASHION CHEMICALS, GMBH & CO KG, Geretsried (DE)

(21) Appl. No.: 13/398,698

(22) Filed: Feb. 16, 2012

Related U.S. Application Data
(60) Provisional application No. 61/443,619, filed on Feb. 16, 2011.

(51) Int. Cl. A01N 25/02; A01P 3/00; A01P 7/04; A01P 13/00; B32B 21/04; A01H 5/00; A01K 67/00; C10M 169/04; B32B 17/06; B32B 9/00; H01B 1/12; A01P 21/00

(52) U.S. Cl. 800/8; 514/772; 252/500; 504/362; 800/298; 508/304; 71/27; 504/101; 428/426; 428/688; 428/537.1

(57) ABSTRACT

Embodiments include adjuvant formulations containing an alkoxylated alkanol adjuvant having the following alkanol structure:

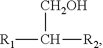

where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof. Further embodiments included methods for making and using the alkoxylated alkanol adjuvant formulations.

### CROSS REFERENCE TO RELATED APPLICATIONS

The present application claims the benefits of U.S. Provisional Application Serial No. 61/443,619 filed Feb. 16, 2011 entitled “GREEN LEAF FULLCOTE”, the entire contents of which is incorporated herein by this reference.

### FIELD OF INVENTION

Embodiments include adjuvant formulations containing an alkoxylated non-ionic alkanol adjuvant having the following alkanol structure:

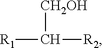

where total number of carbon atoms of R₁ and R₂ is from about 3 to about 36, R₂ can be hydrogen or C₁-C₁₈, and R₁ may or may not be branched.

### BACKGROUND OF THE INVENTION

In general, an adjuvant is a substance that enhances one or more of efficacy, wetting, spreading, and uptake of an active agent, in particular the efficacy, wetting, spreading, and uptake of an active agent contained in an aqueous solution. The active agent may be a lubricant, anti-static agent, scouring agent, herbicide, insecticide, fungicide, foliar nutrient, plant growth regulator, or such. The target substrate may be a plant, a hard surface or a fiber.

### SUMMARY OF THE INVENTION

These and other needs are addressed by the various embodiments and configurations.

Some embodiments include an aqueous slurry composition containing an active agent and an alkoxylated alkanol adjuvant having from about 1 to about 30 moles of alkoxylate, the alkanol having the following structure:

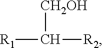

where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof.

Preferably, the alkoxylated alkanol adjuvant has from about 1 to about 5 moles of the alkoxylate, more preferably from about 1 to about 3 moles of the alkoxylate. Even more preferably, the alkoxylated alkanol adjuvant has about 2 moles of the alkoxylate. The alkoxylate may be ethylene oxide, propylene oxide or a mixture of ethylene oxide and propylene oxide.

In some formulations, the total number of carbon atoms of R₁ and R₂ may be from about 3 to about 18. In some embodiments, R₂ is CH₃ and R₁ contains at least two branch sites.

In some embodiments, the alkanol comprises one of a C₁₂, C₁₃, C₁₄, C₁₅, C₁₆, C₁₇, C₁₈ alkanol or mixture thereof. Preferably, the alkanol is a C₁₂-C₁₈ alkanol having about 2 moles of ethylene oxide per mole of the C₁₂-C₁₈ alkanol. More preferably, alkoxlyated alkanol comprises isostearyl alcohol, more preferably isostearyl alcohol having two moles ethylene oxide.

The active agent bay one of a lubricant, anti-static agent, scouring agent, herbicide, insecticide, fungicide, foliar nutrient, plant growth regulator or a mixture thereof.

Preferably, the aqueous slurry composition contains no more than about 1 wt % of the alkoxylated alkanol adjuvant, more preferably the aqueous slurry composition contains from about 0.25 wt % to about 0.5 wt % of the alkoxylated alkanol adjuvant.

Some embodiments include a method for treating a plant by applying to a least a portion of a surface of a plant an effective amount of a slurry composition containing an active agent and an alkoxylated alkanol adjuvant having from about 1 to about 30 moles of alkoxylate, the alkanol having the following structure:

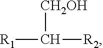

where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof.

Preferably, the alkoxylated alkanol adjuvant has from about 1 to about 5 moles of the alkoxylate, more preferably from about 1 to about 3 moles of the alkoxylate. Even more preferably, the alkoxylated alkanol adjuvant has about 2 moles of the alkoxylate. The alkoxylate may be ethylene oxide, propylene oxide or a mixture of ethylene oxide and propylene oxide.

In some formulations, the total number of carbon atoms of R₁ and R₂ may be from about 3 to about 18. In some embodiments, R₂ is CH₃ and R₁ contains at least two branch sites.

In some embodiments, the alkanol comprises one of a C₁₂, C₁₃, C₁₄, C₁₅, C₁₆, C₁₇, C₁₈ alkanol or mixture thereof. Preferably, the alkanol is a C₁₂-C₁₈ alkanol having about 2 moles of ethylene oxide per mole of the C₁₂-C₁₈ alkanol. More preferably, alkoxlyated alkanol comprises isostearyl alcohol, more preferably isostearyl alcohol having two moles ethylene oxide.

The active agent bay one of a lubricant, anti-static agent, scouring agent, herbicide, insecticide, fungicide, foliar nutrient, plant growth regulator or a mixture thereof.

Preferably, the aqueous slurry composition contains no more than about 1 wt % of the alkoxylated alkanol adjuvant, more preferably the aqueous slurry composition contains from about 0.25 wt % to about 0.5 wt % of the alkoxylated alkanol adjuvant.

Preferably, the plant is selected from the group consisting of fruits, vegetables, grains, legumes, tress, scrubs, flowers, grasses, roots, landscape plants, ornamental plants, and crop plants.

Preferably, the applying includes a process selected from the group consisting of spraying, painting, and dipping. More preferably, the applying further includes treating the plant to substantially protect the plant from pest infestation and/or other damage to the plant. In some embodiments, the treating further includes providing the plant with nutrients.

In some embodiments, the applying of the slurry composition further includes applying the slurry to one of a first and second surface portion of the plant, wherein, thereafter the applying of the slurry, the slurry spreads and/or wets the other of the first and second surface portions.

Some embodiments include a coated non-fluid substrate having at least a portion of the non-fluid substrate surface is coated with an aqueous slurry composition containing an active agent and an alkoxylated alkanol adjuvant having from about 1 to about 30 moles of alkoxylate, the alkanol having the following structure:

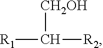

where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof.

Preferably, the alkoxylated alkanol adjuvant has from about 1 to about 5 moles of the alkoxylate, more preferably from about 1 to about 3 moles of the alkoxylate. Even more preferably, the alkoxylated alkanol adjuvant has about 2 moles of the alkoxylate. The alkoxylate may be ethylene oxide, propylene oxide or a mixture of ethylene oxide and propylene oxide.

In some formulations, the total number of carbon atoms of R₁ and R₂ may be from about 3 to about 18. In some embodiments, R₂ is CH₃ and R₁ contains at least two branch sites.

In some embodiments, the alkanol comprises one of a C₁₂, C₁₃, C₁₄, C₁₅, C₁₆, C₁₇, C₁₈ alkanol or mixture thereof. Preferably, the alkanol is a C₁₂-C₁₈ alkanol having about 2 moles of ethylene oxide per mole of the C₁₂-C₁₈ alkanol. More preferably, alkoxlyated alkanol comprises isostearyl alcohol, more preferably isostearyl alcohol having two moles ethylene oxide.

The active agent bay one of a lubricant, anti-static agent, scouring agent, herbicide, insecticide, fungicide, foliar nutrient, plant growth regulator or a mixture thereof.

Preferably, the aqueous slurry composition contains no more than about 1 wt % of the alkoxylated alkanol adjuvant, more preferably the aqueous slurry composition contains from about 0.25 wt % to about 0.5 wt % of the alkoxylated alkanol adjuvant. Preferably, the non-fluid substrate is one of earth, a flooring material, a table, a counter top, a fiber, a fiber containing material, a plant or plant product, a plant by-product, an animal, an animal by-product, a polymeric material, a natural occurring material, a metal or metal containing material, glass, wood, a composite material, a ceramic, or combinations and mixtures thereof.

Some embodiments include a method for treating a non-fluid substrate by contacting to a portion of a non-fluid substrate surface with an aqueous slurry composition containing an active agent and an alkoxylated alkanol adjuvant having from about 1 to about 30 moles of alkoxylate, the alkanol having the following structure:

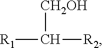

where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof, and thereafter the contacting of the slurry with the non-fluid surface, the slurry spreads and/or wets a substantial portion of the non-fluid substrate not contacted with the aqueous solution.

Preferably, the alkoxylated alkanol adjuvant has from about 1 to about 5 moles of the alkoxylate, more preferably from about 1 to about 3 moles of the alkoxylate. Even more preferably, the alkoxylated alkanol adjuvant has about 2 moles of the alkoxylate. The alkoxylate may be ethylene oxide, propylene oxide or a mixture of ethylene oxide and propylene oxide.

In some formulations, the total number of carbon atoms of R₁ and R₂ may be from about 3 to about 18. In some embodiments, R₂ is CH₃ and R₁ contains at least two branch sites.

In some embodiments, the alkanol comprises one of a C₁₂, C₁₃, C₁₄, C₁₅, C₁₆, C₁₇, C₁₈ alkanol or mixture thereof. Preferably, the alkanol is a C₁₂-C₁₈ alkanol having about 2 moles of ethylene oxide per mole of the C₁₂-C₁₈ alkanol. More preferably, alkoxlyated alkanol comprises isostearyl alcohol, more preferably isostearyl alcohol having two moles ethylene oxide.

The active agent bay one of a lubricant, anti-static agent, scouring agent, herbicide, insecticide, fungicide, foliar nutrient, plant growth regulator or a mixture thereof.

Preferably, the aqueous slurry composition contains no more than about 1 wt % of the alkoxylated alkanol adjuvant, more preferably the aqueous slurry composition contains from about 0.25 wt % to about 0.5 wt % of the alkoxylated alkanol adjuvant. Preferably, the non-fluid substrate is one of earth, a flooring material, a table, a counter top, a fiber, a fiber containing material, a plant or plant product, a plant by-product, an animal, an animal by-product, a polymeric material, a natural occurring material, a metal or metal containing material, glass, wood, a composite material, a ceramic, or combinations and mixtures thereof.

These and other advantages will be apparent from the disclosure contained herein.

As used herein, the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The preceding is a simplified summary of the disclosure to provide an understanding of some aspects of the disclosure. This summary is neither an extensive nor exhaustive overview of the disclosure and its various embodiments. It is intended neither to identify key or critical elements of the disclosure nor to delineate the scope of the disclosure but to present selected concepts of the disclosure in a simplified form as an introduction to the more detailed description presented below. As will be appreciated, other embodiments of the disclosure are possible utilizing, alone or in combination, one or more of the features set forth above or described in detail below.

### DETAILED DESCRIPTION

### The Adjuvant

Broadly, adjuvants according various embodiments comprise alkoxylated alkanols. The alkanol comprising the alkoxylated alkanol adjuvant is depicted by the following generalized chemical formula:

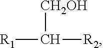

where the total number of carbon atoms of R₁ and R₂ is from about 3 to about 36, preferably from about 9 to about 28. More preferably, the total number of carbon atoms of R₁ and R₂ is from about 12 to about 18. The R₁ group may or may not have a branching site. Preferably, the R₁ group comprises from about C₁ to about C₁₈ carbon chain. In some embodiments, the R₂ group may be hydrogen or from about C₁ to about C₁₈ carbon chain. Preferably, when the R₂ is a methyl group the R₁ group is a highly branched chain having at least two branch sites. In some embodiments, the R₁ group is linear chain lacking a branching site. Preferably, the R₁ group has at least one branch site, more preferably at least two branch sites. In accordance with some embodiments, the R₂ group is one of at least a C₃ group or a C₅ group with the balance of the carbon atoms existing in R₁. Moreover, the R₂ group may or may not contain a branching site. A non-limiting example of an alkanol is a β-branched alkanol.

In some embodiments, the total number of carbon atoms of R₁ and R₂ in the branched and/or un-branched alkanol is from about 1 to about 20. In other embodiments, the total number of carbon atoms of R₁ and R₂ in the adjuvant is from about 1 to about 18. When present, the branching is typically on the main carbon backbone. The branch may be, but is not limited to, a methyl branch.

Branched alkanols can be formed in any manner and/or process for making a branched alkanol. Non-limiting examples of suitable alkanols are:

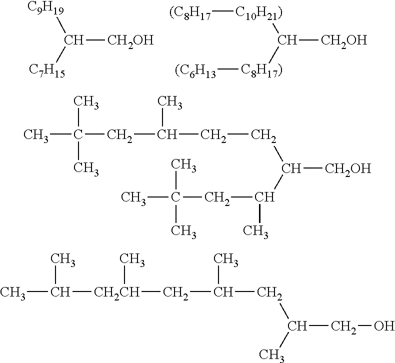

The alkoxylated alkanol adjuvant may comprise a branched alkanol, un-branched alkanol or mixture of branched and un-branched alkanols. The alkoxylated alkanol adjuvant commonly has from about 1 to about 30 moles of alkoxylate, more commonly from about 1 to about 20 moles of alkoxylate, even more commonly from about 1 to about 10 moles of alkoxylate, yet even more commonly from about 1 to about 5 moles of alkoxylate, still yet more commonly from about 1 to about 4 moles of alkoxylate, still yet more commonly from about 1 to about 3 moles of alkoxylate, or yet still more commonly from about 1 to about 2 moles of alkoxylate. Preferably, the alkoxylated alkanol adjuvant has no more than about 4 moles of alkoxylate, more preferably no more than about 3 moles of alkoxylate. Even more preferably, the alkoxylated alkanol adjuvant has no more than about 2 moles of alkoxylate.

In accordance with some embodiments, the alkoxylated alkanol adjuvant has from about 1 to about 8 moles of ethylene oxide per mole of alkanol. The alkoxylated alkanol adjuvant preferably has from about 1 to about 3 moles of ethylene oxide per mole of alkanol, more preferably about 2 moles of ethylene oxide per mole of alkanol. In some embodiments, the alkoxylated alkanol adjuvant is formed from a mixture of ethylene oxide and propylene oxide.

In one embodiment, the alkoxylated alkanol adjuvant comprises a C₁₂-C₁₈ alkanol or mixture thereof having from 1 to about 3 moles of ethylene oxide. Preferably, the alkoxylated alkanol adjuvant comprises a C₁₂-C₁₈ alkanol having about 2 moles ethylene oxide.

In some embodiments, the alkoxylate is a mixture of ethylene oxide and propylene oxide. The molar ratio of ethylene oxide to propylene oxide can be from about 10:1 to about 1:10, from about 7:1 to about 1:7, from about 5:1 to about 1:5, from about 3:1 to about 1:3, from about 2:1 to about 1:2, or about 1:1.

In some embodiments, the alkoxylated alkanol adjuvant comprises isostearyl alcohol having two moles ethylene oxide. It is believed that the isostearyl alcohol comprises primarily the methyl branched series or an alkanol having chemical structure generally resembling:

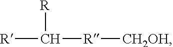

where R is methyl and the location of the R preferably varies in the 2 to 18 position, more preferably varies in the 2 to 10 position of the stearyl backbone.

The alkoxylated alkanol adjuvant can comprise, in some embodiments, a mixture of branched alkanols and one or more of their alkoxylates. Mixtures of branched alkanols and their alkoxylates are particularly effective as alkoxylated adjuvants. The amount of alkoxylation relative to the branched alkanol typically increases at low molar ratios of alkylene oxide to the branched alkanol.

The alkanol may comprise one of a C₁₂, C₁₃, C₁₄, C₁₅, C₁₆, C₁₇, C₁₈ alkanol or mixture thereof. In some formulations, the adjuvant may comprise one or more C₁₂ to C₁₈ alkanol commonly having about 1 mole of mole of ethylene oxide per mole of aklanol, more commonly about 1.5 moles of ethylene oxide per mole of aklanol, even more commonly about 2 moles of ethylene oxide per mole of aklanol, yet even more commonly about 2.5 moles of ethylene oxide per mole of aklanol, or still yet even more commonly about 3 moles of ethylene oxide per mole of aklanol.

In some embodiments, the adjuvant comprises one of stearyl alcohol, isostearyl alcohol or a mixture thereof. Preferably, the adjuvant comprises isostearyl alcohol. More preferably, the adjuvant comprises isostearyl alcohol having one of about 0.2 moles of ethylene oxide per mole of isostearyl alcohol and/or alkoxylated alkanol adjuvant, about 0.5 moles of ethylene oxide per mole of isostearyl alcohol and/or alkoxylated alkanol adjuvant, about 1 mole of ethylene oxide per mole of isostearyl alcohol and/or alkoxylated alkanol adjuvant, about 1.5 moles of ethylene oxide per mole of isostearyl alcohol and/or alkoxylated alkanol adjuvant, about 2 moles of ethylene oxide per mole of isostearyl alcohol and/or alkoxylated alkanol adjuvant, about 2.5 moles of ethylene oxide per mole of isostearyl alcohol and/or alkoxylated alkanol adjuvant, and about 3 moles of ethylene oxide per mole of isostearyl alcohol and/or alkoxylated alkanol adjuvant.

In some embodiments, the alkoxylated alkanol adjuvant is substantially biodegradable. More preferably the alkoxylated alkanol adjuvant is substantially free of one or both of fluorine and silicone.

Adjuvant-Containing Formulations

Some embodies include an aqueous slurry and/or emulsion composition and/or formulation containing an active agent and an alkoxylated alkanol adjuvant. In some embodiments, the slurry composition typically contains no more than about 5 wt % of the alkoxylated alkanol adjuvant, more typically no more than about 4 wt % of the alkoxylated alkanol adjuvant, even more typically no more than about 3 wt % of the alkoxylated alkanol adjuvant, yet even more typically no more than about 2 wt % of the alkoxylated alkanol adjuvant, still yet even more typically no more than about 1 wt % of the alkoxylated alkanol adjuvant, still yet even more typically no more than about 0.5 wt % of the alkoxylated alkanol adjuvant, or yet still even more typically no more than about 0.25 wt % of the alkoxylated alkanol adjuvant. In some embodiments, the slurry composition commonly contains from about 0.1 to about 5 wt % of the alkoxylated alkanol adjuvant, more commonly from about 0.1 to about 4 wt % of the alkoxylated alkanol adjuvant, even more commonly from about 0.1 to about 3 wt % of the alkoxylated alkanol adjuvant, yet even more commonly from about 0.25 to about 2 wt % of the alkoxylated alkanol adjuvant, still yet even more typically from about 0.25 wt % to about 1 wt % of the alkoxylated alkanol adjuvant, or yet still from about 0.25 to about 0.5 wt % of the alkoxylated alkanol adjuvant. However, in some embodiments, slurry compositions can be prepared with lower levels of the alkoxylated alkanol adjuvant, preferably with from about 0.01 wt % to about 5 wt % of the alkoxylated alkanol adjuvant, more preferably from about 0.05 to about 2.5 wt % of the alkoxylated alkanol adjuvant, or even more preferably from about 0.1 to about 1.5 wt % of the alkoxylated alkanol adjuvant.

In some embodiments, the alkoxylated alkanol adjuvant formulation comprises an aqueous slurry and/or emulsion of water and one or more agricultural agents to form an agricultural spray formulation. The agricultural agent can be one of an herbicide, insecticide, fungicide, foliar nutrient, plant growth regulator or mixture thereof. In some embodiments, the agricultural spray formulation may also include other agents for enhancing fluidity, reducing foam formation, or such.

Non-limiting examples of suitable herbicides are 2,4-D, aminopyralid, atrazine, clopyralid, dicamba, glufosinate ammonium, fluoroxypyr, glyphosate, imazapyr, imazapic, linuron, metolachlor, paraquat, pendimethalin, picloram, sodium chlorate, trichlopyr, 2,4,5-T, agent orange, agent purple and agent pink.

Non-limiting examples of suitable insecticides are: organochlorides (such as, DDT, aldrin, chlordane, chlordecone, dieldrin, endosulfan, endrin, heptachlor, hexachlorobenze, lindane, methoxychlor, mirex, pentachlorophenol, TDE to name a few) organophosphates (such as, acephate, aziphos-methyl, bensulide, chlorethoxyfos, chlorpyrifos, chlorpyriphos-methyl, diazinon, dichlorvos, dicrotophos, dimethoate, disulfoton, ethoprop, fenamiphos, fenitrothion, fenthion, fosthiazate, malathion, methamidophos, methidathion, mevinphos, monocrotophos, naled, omethoate, oxydemeton-methyl, parathion, parathion-methyl, phorate, phosalone, phosmet, phostebupirim, phoxim, pirimiphos-methyl, profenofos, terbufos, tetrachlorvinphos, tribufos, trichlorfon and such), carbamates (such as aldicarb, bendiocarb, carbofuran, carbaryl, dioxacarb, fenobucarb, fenoxycarb, isoprocarb, methomyl, 2-(1-methylpropyl)phenyl methylcarbamate), pyrethroids (such as, allethrin, bifenthrin, cyhalothrin, lambda-cyhalothrin, cypermethrin, cyfluthrin, deltamethrin, etofenprox, fenvalerate, permethrin, phenothrin, prallethrin, resmethrin, tetramethrin, trlomethrin, transfluthrin and so forth), neonicotinoids (such as, acetamiprid, clothianidin, imidacloprid, nitenpyram, nithiazine, thiaclopid, thiamethoxam and such), ryanoids (such as, rynaxypyr), growth regulators (such as, methoprene, hydroprene, biflubenzuron, tebufenoxide, and such), plant derived (anabasine, anethole, annonin, asimina, azadirachtin, caffeine, carapa, cinnamaldehyde, cinnamon leaf oil, cinnamyl acetate, citral, deguelin, derris, rotenone, eugenol, linalool, myristicin, neen, nicotine, oregano oil, polyketide, pyrethrum, pyanodine, tetranortriterpenoid, thymol and such), diflubenzurons, hydroprenes, kinoprenes, methoprene, tebufenozides, and polygodials,

Non-limiting examples of suitable fungicides are tea tree oil, cinnamaldehyde, jojoba oil, neem oil, rosemary oil, monocerin, milk, ampelomyces quisqualis, vinclozolin, azoles, carbendazim, diethofencarb, dicarboximide, phenylamide, metalazyl and so forth.

Non-limiting examples of suitable foliar nutrients are nitrogen, phosphorous, potassium, magnesium, calcium, sulfur, iron, zinc, boron, copper, molybdenum, manganese, and chlorine.

In accordance with some embodiments, an effective amount of the adjuvant is mixed with water and the one or more agricultural agents.

The use of adjuvants when spraying pesticides on agricultural crops is a common practice that serves to improve the performance of pesticides by helping to achieve uniform distribution of the pesticides on leaf and fruit surfaces, reduce pesticide runoff and redistribute pesticides following rain events, and aid in the movement of systemic pesticides into leaf cells, including vascular tissue. Some adjuvants may also exhibit direct toxicity to certain pests.

In some embodiments, the alkoxylated alkanol adjuvant formulations include slurry and/or emulsion compositions for applying one or more of pesticides, rodenticides, herbicides, algicides, fungicides, mold killing agents on plants and/or other substrates. The other substrates can include hard surfaces, soft surfaces, and/or pliable surface to name a few. Furthermore, the alkoxylated alkanol adjuvant can be used to in formulations, such as an aqueous slurry and/or emulsion composition for applying one or more of lubricant, anti-static agent, anti-glare agent, scouring agent, herbicide, fungicide, foliar nutrient, and plant growth regulator polishing agent, functional coating, decorative coating, paints or such to any one or more of the above identified substrates. It can be appreciated that the substrate may comprise a polymeric material, a natural occurring material (such as a plant, any product produced by a plant, wood, glass, a plant by product or animal or any animal by product), a metal or metal containing material, a composite material, a ceramic, or combinations and mixtures thereof.

Treatments Using the Adjuvant Some embodiments include a method for treating a plant, comprising contacting at least a portion of a surface of the plant an effective amount of a slurry and/or emulsion composition comprising the alkoxylated alkanol adjuvant, preferably the slurry and/or emulsion composition comprises the alkoxylated alkanol adjuvant and an active agent. The active agent may preferably comprise one or more of the above identified active agents. It can be appreciated that term “plant” refers to one or more the plant, products produced by the plant (such as, its fruit, flower to name a few), and/or by-products.

The method may further include applying the slurry and/or emulsion composition by a process selected from the group consisting of spraying, painting, and dipping. The plant may be selected from the group consisting of fruits, vegetables, grains, legumes, tress, scrubs, flowers, grasses, roots, landscape plants, ornamental plants, and crop plants. The treating of the plant may further include protecting the plant from pest infestation. In some embodiments, the method may further include applying the slurry and/or emulsion composition substantially to one of a first and second surface portion of the plant, and, thereafter applying the slurry, the slurry spreads and/or wets the other of the first and second surface portions.

Some embodiments include a non-fluid substrate having at least a portion of the substrate surface coated with the alkoxylated alkanol adjuvant, such as but not limited to an aqueous slurry and/or emulsion comprising the alkoxylated alkanol adjuvant.

Some embodiments include a method for applying an active to a first portion of a surface by contacting a second portion of the surface with a fluid contacting the active and an effective amount of the alkoxylated alkanol adjuvant. An effective amount of the alkoxylated alkanol adjuvant is substantially enough alkoxylated alkanol adjuvant for the fluid to spread and wet the non-parallel adjoining surface. Preferably, the first and second portions of the surface are non-parallel adjoining surfaces. In some embodiments, the first and second portions of the surface are substantially perpendicular. More preferably, the first and second portions of the surface are in an opposing relationship. Non-limiting examples of the first and second portions of a surface, are: a) the first portion being the top-side of a leaf and second portion being the opposing, bottom-side of the leaf; b) the first portion being a first hard surface (such as, but not limited to a floor) and the second portion being a second hard surface, interconnected and substantially perpendicular to the first hard surface (such as, but not limited to a wall); c) the first and second portions being areal portions of a curved surface, such as, but not limited to first and second areal portions of a surface substantially resembling a sphere (such as a tomato or apple).

### EXAMPLES

Examples A-C summarize an evaluation of alkoxylated branched alkanols as an adjuvant for fruit and vegetable crops, specifically for apple, cabbage and tomato crops having different pests. Within each crop, experiments were designed to compare performance of an alkoxylated branched alkanol adjuvant with the commercial standard adjuvant Kinetic® (a registered trademark of Helena Chemical Co. believed to be a proprietary blend of polyalkyleneoxide modified polydimethylsiloxane and nonionic surfactants). The Kinetic® adjuvant is an organo-silicone surfactant comprising a proprietary blend of polyalkyleneoxide modified polydimethylsiloxane and polyoxpropylene-polyoxyethylene block copolymers. The alkoxylated branched alkanol adjuvant was applied alone to detect any potential activity against arthropod pests. The specific insecticides used with adjuvant and Kinetic® varied during the course of the season depending on the pest(s) targeted at each spraying.

### Example A

On 10 May, six-week old ‘Bravo’ cabbage transplants were set on plots. The plots had beds spaced 5 feet apart with double-rows of cabbage. The plots consisted of 25-foot long rows with two rows of cabbage spaced 15 inches apart within and between the rows. Each treatment was replicated four times in a randomized complete block design study. Treatments consisted of the alkoxylated branched alkanol adjuvant at 0.25 and 0.5% v/v with an insecticide, Kinetic® (0.25%) plus insecticide, the alkoxylated branched alkanol adjuvant alone at 0.5%, Kinetic® alone at 0.5%, and a non-treated control. Applications were made on 23 and 30 May, 6, 13, 22 and 29 June, and 6 and 13 July (see Tables 1-10 for insecticides applied). Treatment applications were made with a carbon dioxide powered backpack sprayer delivering materials through two hollow cone nozzles per row at 35 PSI. The 23 May application was made at 25 gallons per acre, and all subsequent applications were made at 50 GPA. With the exception of first three applications, all treatment solutions with the alkoxylated branched alkanol adjuvant were mixed with a high-speed mixer for one minute immediately before applying treatments to plants.

Tobacco thrips populations were monitored. The tobacco thrips populations were dislodged from the plants by gently beating the plants. The dislodged tobacco thrips were collected on a white laminated piece of paper. The number of tobacco thrips collected from 5 plants determined the tobacco thrips population.

Flea beetle populations were assessed. Flea beetle populations were the number flea beetles observed on 10 plants per plot.

Populations of lepidopteran larvae, including cabbage looper, imported cabbageworm, diamondback moth, and cross-striped cabbageworm, were monitored by counting the respective number on 10 plants per plot.

Cabbage heads were harvested on 20 July. The cabbage heads were assessed for marketable yield by rating 20 heads per plot on a scale of 0 to 4, where 0 means no damage, 1 means slight damage to frame leaves, 2 means slight feeding damage to wrapper leaves, 3 means feeding damage to the head, and 4 means severe feeding damage. Non-marketable cabbages heads were cabbage heads having a rating greater than 2. All data were subjected to two-way ANOVA, and LSD (P=0.05) separated the mean values.

Early season thrips populations were high prior to the 23 May application of treatments, and all treatments significantly reduced thrips populations within two days (see Table 1, compare 23 May and 25 May thrips populations). Treatments with Warrior® (registered trademark of Syngenta Crop Protection, Inc., believed to comprise lamba-cyhalothrin, [1a(S*), 3a(Z)]-(±)-cyano-(3-phenoxylphenyl)methyl-3(2-cholor-3,3,3,-trifluoro-1-propenyl)-2,2-dimethylcyclopropanecarboxylate) were most effective, but both with the alkoxylated branched alkanol adjuvant and Kinetic® at 0.5% exhibited thrips activity and significantly reduced numbers below the control. Flea beetle populations were quite low, with significant differences on 30 June and season total numbers (Table 1). Flea beetle counts were highest in the treatments lacking insecticides, and, beetle counts were significantly higher in 0.5% of the alkoxylated branched alkanol adjuvant alone when compared to the control. Season total lepidopteran larval populations (i.e., counts totaled from samples on 9, 16, 24 and 30 June) were relatively low. Imported cabbageworm having the most abundant lepidopteran lava populations. Treatments with insecticide had significantly lower lepidopteran larval populations compared with other treatments. Insecticide performance for the alkoxylated branched alkanol and Kinetic® adjuvant treatments did not significantly differ. Among the alkoxylated branched alkanol and Kinetic® adjuvant treatments lacking an insecticide, imported cabbageworm populations were significantly higher in 0.5% alkoxylated branched alkanol adjuvant versus the 5% Kinetic®, neither of which differed from the control (Table 2). The quality and marketability of cabbage heads were affected only by the applications that included an insecticide. The treatments having the insecticide had better quality and marketability ratings. Lower quality and marketability ratings were observed for the control and treatments comprising the alkoxylated branched alkanol and Kinetic® adjuvants alone.

### Example B

On 19 May, six-week old ‘Red Defender’ tomato transplants were set on plots. The plots consisted of black plastic mulch with drip irrigation, and single 25-feet rows on 5-feet centers. Treatment rows were separated by non-treated rows. The tomato plants were spaced 1.5 feet apart within rows. The tomato plants were staked and strung as needed and sprayed with a standard fungicide program.

A randomized complete block design study was used to set-up the treatment program. The treatments were replicated four times. Treatments consisted of the alkoxylated branched alkanol adjuvant at 0.25 and 0.5% v/v with an insecticide, Kinetic® (0.25%) with an insecticide, the alkoxylated branched alkanol adjuvant alone at 0.5%, Kinetic® alone at 0.5%, and a non-treated control.

Treatment solutions containing the alkoxylated branched alkanol adjuvant were mixed with a high-speed mixer for 1 minute immediately before application to the tomato plants. All treatments were applied weekly from 10 June to 12 August, for a total of 10 treatment applications.

Treatment applications were made with a carbon dioxide powered backpack sprayer. The sprayer delivered the treatment materials through a spray wand at 35 PSI. The first four treatment applications were made at 50 GPA (two hollow cone nozzles/side/row). The last six applications were made at 75 GPA (three hollow cone nozzles/side/row).

The insecticides included Asana® XL (registered trademark of Dupont, believed to be esfenvalerate, (S)-cyano (c-phenoxyphenyl) methyl (S)-4-chloro-alpha-(1-methylethyl) benzenacetate), Coragen® (registered trademark of Dupont, believed to be chlorantraniliprole, 3-bromo-N-[4-chloro-2-methyl-6-[(methylamino)carbonyl]phenyl]-1-(3-chloro-2-pyridinyl)-1H-pyrazole-5-carboxamide), Radiant® (registered trademark of Dow ArgoSciences believed to be a mixture of spinetoram-J and spinetoram-1, (2R,3aR,5aR,5bS,9S,13S,14R,16aS,16bR)-13-{[(2R,5S,6R)-5-(dimethylamino)-6-methyltetrahydro-2H-pyran-2-yl]oxy}-9-ethyl-14-methyl-7,15-dioxo-2,3,3a,4,5,5a,5b,6,7,9,10,11,12,13,14,15,16a,16b-octadecahydro-1H-as-indaceno[3,2-d]oxacyclododecin-2-yl 6-deoxy-3-O-ethyl-2,4-di-O-methyl-alpha-L-mannopyranoside and (2S,3aR,5aS,5bS,9S,13S,14R, 16aS,16bS)-13-{[(2R,5S,6R)-5-(dimethylamino)-6-methyltetrahydro-2H-pyran-2-yl]oxy}-9-ethyl-4,14-dimethyl-7,15-dioxo-2,3,3a,5a,5b,6,7,9,10,11,12,13,14,15,16a,16b-hexadecahydro-1H-as-indaceno[3,2-d]oxacyclododecin-2-yl 6-deoxy-3-O-ethyl-2,4-di-O-methyl-alpha-L-mannopyranoside) and Warrior® 1CS (registered trademark of Syngent believed to be lambdacyhalothrin). The Asana® XL treatments (6 fl oz/A) were on 10 and 17 June, the Coragen treatments (4 fl oz/A) were on 24 June. 15 July and 5 Aug, the Radiant treatments (6 fl oz/A) were on 1 and 22 July and 12 Aug, and the Warrior® 1CS (2.56 fl oz/A) treatments were on 8 and 29 July.

The miticide was Oberon® 4SC (registered trademark of Bayer CropScience, believed to be spiromesifen, 2-oxo-3-(2,4,6-trimethylphenyl)-1-oxaspiro[4.4]non-3-3-en-4-yl 3,3-dimethylbutanoate). The Oberon® miticide (7 fl oz/A) was tank mixed with Radiant and applied on 22 July.

Flower thrips populations were monitored. The flower thrips populations were determined by harvesting ten flowers per plot, placing the harvested flowers in a vial having 50% aqueous ethanol solution, and counting dislodged insects. The insect count was conducted using a stereomicroscope. The flower thrips populations were of moderate intensity. Significant population differences occurred on 16-June, when all treatments, except 0.5% Kinetic® alone, reduced the flower thrips populations below the control (Table 3). The 0.25% alkoxylated branched alkanol adjuvant with Insecticide and 0.25% Kinetic® with insecticide had the lowest season total cumulative flower thrips populations. The flower thrips populations for tomato plants treated with the 25% alkoxylated branched alkanol adjuvant with Insecticide and 0.25% Kinetic® with insecticide were statistically equivalent.

Potato aphid populations were determined by counting the total number of aphids on ten recently, fully expanded leaves per plot. Potato aphid populations were first detected on 14 July, and increased from about two potato aphids per leaf to about fourteen per leaf by 18 August (Table 4). For most treatment days, treatments that included insecticides had significantly lower potato aphid populations than the control or the treatments comprising the alkoxylated branched alkanol or Kinetic® adjuvants alone.

Two-spotted spider mite populations were determined by counting the number of two-spotted mites on ten terminal leaflets per plot. Two-spotted spider mite populations were high. The two-spotted spider mite population increased from an average in the control of about 0.5 mites per leaf (on 21 July) to about eighteen per leaf (on 18 August). Statistically, compared to the control, the two-spotted spider mite populations did not differ among treatments evaluated. However, the lowest two-spotted spider mite population was observed for the alkoxylated branched alkanol adjuvant (0.5%) with insecticide treatment (Table 5).

Damage to fruit by lepidopteran larvae (primarily tomato fruit worm) averaged about 7% in the control. The alkoxylated branched alkanol and Kinetic® adjuvant treatments alone did not significantly reduce the level of lepidopteran larvae damage below the control (Table 6). The lowest lepidopteran larvae damage level observed was for tomatoes treated with an insecticide and using one of the alkoxylated branched alkanol (0.25%) and Kinetic® (0.5%) adjuvants, each of which significantly reduced lepidopteran damage below the control.

Stinkbug damage was high, averaging almost 30% in the control. The alkoxylated branched alkanol and Kinetic® adjuvant treatments alone (that is, without an active) did not significantly reduce the level of stinkbug damage below the control. Treatments that included an insecticide significantly reduced stinkbug damage. Referring to stinkbug damage on the 18 August harvest day, damage in the tomatoes treated with insecticide and 0.25% alkoxylated branched alkanol adjuvant (0.25%) had significantly less stinkbug damage than tomatoes treated with the same insecticide and alkanol adjuvant at 0.5%.

Mature tomatoes were harvested from the eight middle plants of each plot on 28 Jul and 11 and 18 Aug. Each tomato was graded for insect damage. The data were subjected to two-way analysis of variance between groups, with a least significant difference of P=0.05 separating the mean values of the groups.

### Example C

Treatment trials were conducted in a mature block of ‘Golden Delicious’ apple trees spaced 10-ft apart within rows having 25-foot centers and an estimated tree-to-row volume of about 250 GPA. Each plot consisted of 2 adjacent trees within a row and at least one non-treated tree separating the treatment plots. Treatment solutions consisted of the alkoxylated branched alkanol adjuvant at 0.25 and 0.5% v/v with an insecticide, Kinetic® at 0.25% v/v with an insecticide, the adjuvant alone at 0.5%, and a non-treated control.

Each treatment was replicated 4 times in a randomized complete block experimental design. Each application was made with a tractor-mounted airblast sprayer. The sprayer delivered 100 GPA with a recycling pump agitating the treatment solutions.

The treatment solutions were applied at the following times: at petal fall on 14 April, 2 and 20 May, 3 June, 8 July and 5 August. The insecticide applied on 14 April was Actara 25WDG (4.5 oz/A). Delegate 25WDG (5.0 oz/A) was applied in the 2 and 20 May treatments, while Assail 30WDG (5 oz/A) was applied in the 3 June treatments. The insecticide Calypso 4SC (6.0 fl oz/A) was applied in the 8 July and 5 August treatments.

Counts of indirect pests, including European red mite, green aphids and potato leafhopper, were timed to coincide with peak densities of these insects. Mites were counted on 10 leaves per plot, leafhoppers on 10 shoots per plot, and green apple aphids by counting the number of aphids on the most infested leaf on each of 10 sprouts per plot.

The apples per plot were harvested on 8 September. Insect damage was determined by recording the number of apples damaged per plot by various insects. All data were subjected to two-way analysis of variance between groups, with a least significant difference of P=0.05 separating the mean values of the groups.

European red mite populations were low. More specifically, the European red mite populations were less than about 1 mite per 10 leaves for any of treatments (see Table 7). The mite populations were generally too low to statistically determine treatment affects.

Potato leafhopper populations were highest in early June. 12 days after the 20 May application of Delegate (on 2 June) leafhopper populations were lower for all insecticide treatments compared to the control and the alkoxylated branched alkanol adjuvant with an insecticide. Leafhopper counts on 8 June (5 days after treatment applications of 3 June) were higher in the control compared to all other treatments, including the alkoxylated branched alkanol adjuvant alone (see Table 8). A similar trend was evident in season total counts of leafhoppers.

Green apple aphid populations were low and variable and exhibited little differences among treatments (see Table 9).

About 21% of the apples exhibited larval entries. The larval entries were typically internal feeding lepidopteran, more typically larval entries were a complex of codling and oriental fruit moths. The alkoxylated branched alkanol adjuvant alone at 0.5% did have numerically lower damage than the control. However, lower damage observed for the alkoxylated branched alkanol adjuvant alone at 0.5% alone may or may not be statistically significant. The most effective treatments were for insecticide treatments applied with an adjuvant.

Comstock mealybug damage varied with the treatment application. Comstock mealybug damage for the alkoxylated branched alkanol adjuvant without insecticide had more damage than all other treatments, including the control. It is believed that higher damage was due to a highly aggregated distribution of mealybugs in the alkoxylated alkanol adjuvant trial site rather than a treatment effect.

Table 11 summarizes Examples A-C test results. Briefly, the alkoxylated alkanol adjuvant is at least as effective if not better, as demonstrated in some instances (see Tables 1-10). Moreover, the alkoxylated alkanol adjuvant is substantially more biodegradable than traditional adjuvants. Biodegradable adjuvants are preferred in agricultural applications.

### Example D

The spreading ability of solutions with and without an alkoxylated alkanol adjuvant on a control substrate were determined The test method consisted of applying 0.1 ml drops of solutions with and without the alkoxylated alkanol adjuvant to substrate and measuring solution coverage due to spreading after a period of time. The area coverage with the solution with the alkoxylated alkanol adjuvant was compared to the area coverage of the solution lacking the alkoxylated alkanol adjuvant.

The first spreading study compared the spreading ability of butyl stearate with and without 1 wt % of the alkoxylated alkanol adjuvant. The substrates evaluated were polyethylene and paraffin coated paper. The butyl stearate solution containing 1 wt % of the alkoxylated alkanol adjuvant had a 25% greater area coverage on polyethylene and 67% greater area coverage on the paraffin coated paper, respectively, due to spreading than the butyl stearate solution lacking the alkoxylated alkanol adjuvant.

The second spreading study compared the spreading ability of a solution comprising 50 parts water and 50 parts PEG600 laurate with and without 0.5 wt % of the alkoxylated alkanol adjuvant. The substrates evaluated were polyamide film, stainless steel and polyester film. The water/PEG600 laurate solution containing 0.5 wt % of the alkoxylated alkanol adjuvant had a 210% greater area coverage on the polyamide film, a 570% greater area coverage on the stainless steel, and 160% greater area coverage on the polyester film, respectively, due to spreading than the water/PEG 600 laurate solution lacking the alkoxylated alkanol adjuvant.

### Example E

The spreading ability of solutions with and without an alkoxylated alkanol adjuvant were determined on a plant. The plants were ornamental “Peace Lily” plants. One plant was treated with an aqueous solution containing 5 wt % of the alkoxylated alkanol adjuvant and 0.01 wt % of an optical brightener. A second plant was treated with a commercial spectracide brand triazicide insecticide (with 0.002% lambdacyhalohtrin) containing 0.25 wt % of the alkoxylated alkanol adjuvant and 0.01 wt % of an optical brighter. A third plant was treated with a commercial spectracide brand triazicide insecticide (with 0.002% lambdacyhalohtrin) and an optical brighter. A fourth plant was not treated and served as the control.

The treatment solutions were applied in three “top down” blasts with a one liter hand-held sprayer. The solutions were applied “top down” in order to apply at least most, if not all, of the treatment solution to the topside of plant's leaves.

Each plant was visually examined under ultra-violet light over various time intervals to document spreading of the applied treatment solutions. The examinations were made under ultra-violet light to take advantage of the fluorescence of the optical brightener added to the treatment solutions. The treatment solutions containing the alkoxylated alkanol adjuvant, compared to the solution lacking the alkoxylated alkanol adjuvant, showed spreading over the entire top surface of the leaf as well as some spreading and/or wrap around of the edge of the leaf to the backside of the leaf.

Compared to the alkoxylated alkanol adjuvant, industry standard wetting agents have been found to produce inadequate wrap around spreading. Most, if not all, of treatment solutions applied to plants are “top down” applications. Plant pests tend to nest and breed on the backside of plant leaves, that is on the side opposing the “top down” treated side. It can be appreciated that, spreading of the agents applied to topside of the leaf to the opposing backside can be advantageous.

TABLE 1
Mean tobacco thrips and flea beetles on cabbage treated with
various adjuvants and insecticides. Mills River, NC. 2011.

| | Thrips/5 plants | | | | Flea beetles/plant | | | | |
|---|---|---|---|---|---|---|---|---|---|
| Treatment* | 20 May | 25 May | 3 Jun | Total | 3 Jun | 9 Jun | 16 Jun | 30 Jun | Total |
| Alkoxylated Alkanol | 35.8ab | 5.5a | 1.0a | 42.3a | 0.0a | 0.2a | 0.2a | 0.4ab | 0.8ab |
| Adjuvant (0.25%) + | | | | | | | | | |
| Insecticide | | | | | | | | | |
| Alkoxylated Alkanol | 41.5ab | 1.8a | 1.8a | 45.0a | 0.1a | 0.1a | 0.3a | 0.8ab | 1.1ab |
| Adjuvant (0.5%) + | | | | | | | | | |
| Insecticide | | | | | | | | | |
| Alkoxylated Alkanol | 33.0ab | 26.5b | 3.8a | 63.3b | 0.1a | 0.2a | 0.4a | 1.9c | 2.5c |
| Adjuvant (0.5%), | | | | | | | | | |
| No Insecticide | | | | | | | | | |
| Kinetic ® (0.25%) + | 49.8b | 2.0a | 1.3a | 53.0a | 0.0a | 0.0a | 0.4a | 0.2a | 0.6a |
| Insecticide | | | | | | | | | |
| Kinetic ® (0.5%), No | 29.8a | 24.8b | 6.8a | 61.3b | 0.1a | 0.1a | 0.6a | 1.0b | 1.7bc |
| Insecticide | | | | | | | | | |
| Control | 36.8ab | 42.5c | 2.3a | 81.5c | 0.1a | 0.1a | 0.1a | 0.6ab | 0.9ab |

Means within the same column followed by the same letter are not significantly different by LSD (P = 0.05).
*Insecticides applied with adjuvant treatments were Warrior ® 1CS (2.56 oz/A) on 23 and 31 May and 13 July, Radiant ® 1SC (6 oz/A) on 31, 6 and 29 June, Coragen ® 1.67SC (4 oz/A) on 13 June, and Avaunt ® 35WDG (3 oz/A) on 22 June and 6 July.

TABLE 2
Mean season total cabbage looper (CL), imported
cabbageworm (ICW), diamondback moth (DBM), and cross-
striped cabbageworm (CSCW) larvae, and cabbage head
quality ratings. Mills River, NC. 2011.

| | | | | | Head quality | |
|---|---|---|---|---|---|---|
| | Mean larvae per 10 plants | | | | Rat- | % |
| Treatment* | CL | ICW | DBM | CSCW | ing | Market. |
| Alkoxylated | 0.3a | 0a | 0a | 0a | 0.2a | 100c |
| Alkanol | | | | | | |
| Adjuvant (0.25%) + | | | | | | |
| Insecticide | | | | | | |
| Alkoxylated | 0.3a | 0.5a | 0a | 0a | 0.1a | 100c |
| Alkanol | | | | | | |
| Adjuvant (0.5%) + | | | | | | |
| Insecticide | | | | | | |
| Alkoxylated | 2.8b | 17.5c | 0.3a | 0.8a | 2.9b | 12.5a |
| Alkanol | | | | | | |
| Adjuvant (0.5%), | | | | | | |
| No Insecticide | | | | | | |
| Kinetic ® | 0a | 0a | 0a | 0a | 0.2a | 100c |
| (0.25%) + | | | | | | |
| Insecticide | | | | | | |
| Kinetic ® | 2.0b | 8.8b | 0.3a | 0a | 3.1b | 2.5a |
| (0.5%), | | | | | | |
| No Insecticide | | | | | | |
| Control | 1.8b | 13.8bc | 0.3a | 0a | 3.3b | 0a |

Means within the same column followed by the same letter are not significantly different by LSD (P = 0.05).
*Insecticides applied with adjuvant treatments were Warrior ® 1CS (2.56 oz/A) on 23 and 31 May and 13 July, Radiant ® 1SC (6 oz/A) on 31, 6 and 29 June, Coragen ® 1.67SC (4 oz/A) on 13 June, and Avaunt ® 35WDG (3 oz/A) on 22 June and 6 July.

TABLE 3
Thrips on flowers of tomato plants treated with various
insecticides and adjuvants. Mills River, NC. 2011.

| | | | | | | | | | Cumulative |
|---|---|---|---|---|---|---|---|---|---|
| Treatment | Rate/A | 16-Jun | 23-Jun | 30-Jun | 7-Jul | 14-Jul | 21-Jul | 11-Aug | thrips days |
| | | Adult thrips per 10 flowers | | | | | | | |
| Insecticide + | 0.25% | 1.5a | 0.5a | 1.0a | 2.3a | 1.0a | 0.0a | 0.8a | 46.4a |
| Alkoxylated | | | | | | | | | |
| Alkanol | | | | | | | | | |
| Adjuvant | | | | | | | | | |
| Insecticide + | 0.50% | 1.8a | 0.3a | 2.0a | 3.3a | 4.3a | 2.8a | 0.0a | 112.9a |
| Alkoxylated | | | | | | | | | |
| Alkanol | | | | | | | | | |
| Adjuvant | | | | | | | | | |
| Alkoxylated | 0.50% | 1.0a | 1.3a | 0.8a | 3.5a | 6.5a | 0.8a | 0.0a | 98.0a |
| Alkanol | | | | | | | | | |
| Adjuvant | | | | | | | | | |
| Insecticide + | 0.25% | 0.5a | 1.0a | 0.8a | 1.0a | 1.5a | 1.0a | 1.0a | 56.0a |
| Kinetic ® | | | | | | | | | |
| Kinetic ® | 0.50% | 1.5a | 2.3a | 0.5a | 2.3a | 6.3a | 2.0a | 0.8a | 119.9a |
| Control | — | 4.3a | 0.5a | 1.8a | 1.8a | 7.5a | 2.0a | 0.8a | 131.3a |
| | | Immature thrips per 10 flowers | | | | | | | |
| Insecticide + | 0.25% | 0.0a | 0.8a | 0.0a | 1.5a | 0.0a | 0.8a | 0.0a | 26.3a |
| Alkoxylated | | | | | | | | | |
| Alkanol | | | | | | | | | |
| Adjuvant | | | | | | | | | |
| Insecticide + | 0.50% | 0.3a | 0.3a | 0.3a | 0.8a | 0.0a | 2.3a | 0.0a | 41.1a |
| Alkoxylated | | | | | | | | | |
| Alkanol | | | | | | | | | |
| Adjuvant | | | | | | | | | |
| Alkoxylated | 0.50% | 0.0a | 0.3a | 0.0a | 2.0a | 0.8a | 1.3a | 0.0a | 38.5a |
| Alkanol | | | | | | | | | |
| Adjuvant | | | | | | | | | |
| Insecticide + | 0.25% | 0.5a | 0.8a | 0.3a | 0.3a | 0.0a | 0.3a | 0.0a | 14.0a |
| Kinetic ® | | | | | | | | | |
| Kinetic ® | 0.50% | 1.3a | 1.0a | 0.0a | 1.5a | 0.8a | 1.8a | 0.0a | 51.6a |
| Control | — | 1.5a | 3.0a | 0.0a | 2.5a | 1.3a | 1.8a | 0.0a | 77.0a |
| | | Total thrips per 10 flowers | | | | | | | |
| Insecticide + | 0.25% | 1.5a | 1.3a | 1.0a | 3.8a | 1.0a | 0.8a | 0.8a | 72.6a |
| Alkoxylated | | | | | | | | | |
| Alkanol | | | | | | | | | |
| Adjuvant | | | | | | | | | |
| Insecticide + | 0.50% | 2.0a | 0.5a | 2.3a | 4.0a | 4.3a | 5.0a | 0.0a | 154.0a |
| Alkoxylated | | | | | | | | | |
| Alkanol | | | | | | | | | |
| Adjuvant | | | | | | | | | |
| Alkoxylated | 0.50% | 1.0a | 1.5a | 0.8a | 5.5a | 7.3a | 2.0a | 0.0a | 136.5a |
| Alkanol | | | | | | | | | |
| Adjuvant | | | | | | | | | |
| Insecticide + | 0.25% | 1.0a | 1.8a | 1.0a | 1.3a | 1.5a | 1.3a | 1.0a | 70.0a |
| Kinetic ® | | | | | | | | | |
| Kinetic ® | 0.50% | 2.8ab | 3.3a | 0.5a | 3.8a | 7.0a | 3.8a | 0.8a | 171.5a |
| Control | — | 5.8b | 3.5a | 1.8a | 4.3a | 8.8a | 3.8a | 0.8a | 208.3a |

Means in the same column followed by the same letter are not significantly different by LSD (p = 0.05).

TABLE 4
Potato aphids on tomato plants treated with various
insecticides and adjuvants. Mills River, NC. 2011.

| | | Aphids per 10 leaflets | | | | | | |
|---|---|---|---|---|---|---|---|---|
| Treatment | Rate/A | 30-Jun | 14-Jul | 21-Jul | 28-Jul | 3-Aug | 11-Aug | 18-Aug |
| Insecticide + | 0.25% | 0.3a | 0.0a | 0.0a | 18.3a | 3.0a | 1.3a | 0.5a |
| Alkoxylated | | | | | | | | |
| Alkanol Adjuvant | | | | | | | | |
| Insecticide + | 0.50% | 0.0a | 1.5a | 4.3a | 1.0a | 9.5a | 0.0a | 10.3b |
| Alkoxylated | | | | | | | | |
| Alkanol Adjuvant | | | | | | | | |
| Alkoxylated | 0.50% | 0.0a | 21.3b | 51.3b | 97.5b | 24.0c | 93.0c | 102.0c |
| Alkanol Adjuvant | | | | | | | | |
| Insecticide + | 0.25% | 0.0a | 0.0a | 2.5a | 1.3a | 0.0a | 0.0a | 2.3ab |
| Kinetic ® | | | | | | | | |
| Kinetic ® | 0.50% | 0.0a | 7.5ab | 71.0b | 72.8b | 7.8ab | 30.8b | 91.0c |
| Control | — | 0.0a | 17.3ab | 67.0b | 51.8b | 45.3d | 85.8bc | 139.5c |

Means in the same column followed by the same letter are not significantly different by LSD (p = 0.05).

TABLE 5
Twospotted spider mites on tomato plants treated with various insecticides and adjuvants. Mills River, NC. 2011.

| | | Mites per 10 leaflets | | | | | | | | |
|---|---|---|---|---|---|---|---|---|---|---|
| Treatment | Rate/A | 21-Jul | 25-Jul | 28-Jul | 1-Aug | 4-Aug | 11-Aug | 15-Aug | 18-Aug | CMD |
| Insecticide + | 0.25% | 22.0a | 18.3a | 12.3a | 9.3a | 11.8a | 89.8a | 134.0a | 261.8a | 1597.1a |
| Alkoxylated Alkanol | | | | | | | | | | |
| Adjuvant | | | | | | | | | | |
| Insecticide + | 0.50% | 10.3a | 2.8a | 15.0a | 17.8a | 19.8a | 49.0a | 118.5a | 200.8a | 1228.9a |
| Alkoxylated Alkanol | | | | | | | | | | |
| Adjuvant | | | | | | | | | | |
| Alkoxylated Alkanol | 0.50% | 2.8a | 14.5a | 36.8a | 31.3a | 28.5a | 71.3a | 128.5a | 232.5a | 1627.1a |
| Adjuvant | | | | | | | | | | |
| Insecticide + | 0.25% | 12.8a | 4.8a | 11.0a | 16.8a | 39.3a | 66.8a | 131.5a | 163.3a | 1407.8a |
| Kinetic ® | | | | | | | | | | |
| Kinetic ® | 0.50% | 40.0a | 5.8a | 21.0a | 28.5a | 19.8a | 85.5a | 134.3a | 176.3a | 1576.6a |
| Control | — | 4.8a | 8.8a | 39.8a | 40.0a | 43.5a | 126.5a | 178.3a | 179.5a | 2125.6a |

Means in the same column followed by the same letter are not significantly different by LSD (p = 0.05).

TABLE 6
Direct damage to tomatoes treated with various insecticides and adjuvants. Mills River, NC. 2011.

| | | % Lep | | | | % Stink bug | | | | % Thrips | | | |
|---|---|---|---|---|---|---|---|---|---|---|---|---|---|
| Treatment | Rate/A | 7-28 | 8-11 | 8-18 | Total | 7-28 | 8-11 | 8-18 | Total | 7-28 | 8-11 | 8-18 | Total |
| Insecticide + | 0.25% | 1.5a | 0.4a | 3.9ab | 1.5ab | 4.0a | 3.4a | 4.7a | 4.2a | 2.7a | 5.0a | 6.0a | 4.3a |
| Alkoxylated Alkanol | | | | | | | | | | | | | |
| Adjuvant | | | | | | | | | | | | | |
| Insecticide + | 0.50% | 3.8a | 4.5ab | 1.5ab | 3.2abc | 4.9ab | 10.0a | 25.3bc | 12.1a | 3.2a | 2.2a | 5.7a | 3.5a |
| Alkoxylated Alkanol | | | | | | | | | | | | | |
| Adjuvant | | | | | | | | | | | | | |
| Alkoxylated Alkanol | 0.50% | 7.5a | 9.9b | 5.4b | 7.8c | 13.3bc | 23.7b | 41.2c | 25.0b | 3.4a | 2.7a | 6.1a | 3.7a |
| Adjuvant | | | | | | | | | | | | | |
| Insecticide + Kinetic ® | 0.25% | 0.0a | 1.9a | 0.0a | 0.9a | 9.4ab | 7.6a | 19.3b | 10.6a | 3.0a | 2.0a | 10.5a | 4.2a |
| Kinetic ® | 0.50% | 3.4a | 7.5b | 2.8ab | 4.6bc | 19.0c | 23.1b | 37.7bc | 26.5b | 7.9a | 6.2a | 5.1a | 5.9a |
| Control | — | 4.9a | 9.4b | 7.5b | 7.0c | 22.5c | 28.6b | 32.4bc | 28.2b | 3.2a | 4.6a | 11.9a | 6.9a |

Means in the same column followed by the same letter are not significantly different by LSD (p = 0.05).

TABLE 7
European red mites on ‘Golden Delicious’ apples treated with different adjuvants. Mills River, NC. 2011.

| | Mites per 10 leaves | | | | | | | |
|---|---|---|---|---|---|---|---|---|
| | | | | | | | | Cumulative |
| Treatment | 10-May | 2-Jun | 8-Jun | 17-Jun | 24-Jun | 1-Jul | 18-Jul | Mite Days |
| Alkoxylated Alkanol Adjuvant (0.25%) + | 0.0a | 0.0a | 0.0a | 0.0a | 0.0a | 0.3a | 0.0a | 3.0a |
| Insecticide | | | | | | | | |
| Alkoxylated Alkanol Adjuvant (0.5%) + | 0.0a | 0.0a | 0.0a | 0.0a | 0.0a | 0.0a | 0.0a | 0.0a |
| Insecticide | | | | | | | | |
| Alkoxylated Alkanol Adjuvant (0.5%), | 0.0a | 0.0a | 0.0a | 0.0a | 0.0a | 0.5a | 0.0a | 6.0a |
| No Insecticide | | | | | | | | |
| Kinetic ® (0.25%) + Insecticide | 0.0a | 0.0a | 0.0a | 0.0a | 0.3a | 0.5a | 0.3a | 9.9a |
| Control | 0.0a | 0.0a | 0.0a | 0.0a | 0.5a | 0.3a | 0.0a | 6.5a |

Means in the same column followed by the same letter are not significantly different by LSD (p = 0.05).

TABLE 8
Potato leafhoppers on ‘Golden Delicious’ apples treated
with different adjuvants. Mills River, NC. 2011.

| | Leafhoppers per 10 shoots | | | | |
|---|---|---|---|---|---|
| | | | | | Season |
| Treatment | 2-Jun | 8-Jun | 17-Jun | 24-Jun | total |
| Alkoxylated Alkanol | 7.8a | 0.0a | 0.5a | 1.3a | 9.5a |
| Adjuvant (0.25%) + | | | | | |
| Insecticide | | | | | |
| Alkoxylated Alkanol | 9.5a | 0.0a | 0.5a | 3.3a | 13.3a |
| Adjuvant (0.5%) + | | | | | |
| Insecticide | | | | | |
| Alkoxylated Alkanol | 6.3a | 0.0a | 3.0a | 3.8a | 13.0a |
| Adjuvant (0.5%), | | | | | |
| No Insecticide | | | | | |
| Kinetic ® (0.25%) + | 15.0c | 0.0a | 1.5a | 0.8a | 17.3a |
| Insecticide | | | | | |
| Control | 14.3c | 4.5b | 2.3a | 9.8a | 30.8b |

Means in the same column followed by the same letter are not significantly different by LSD (p = 0.05).

TABLE 9
Green apple aphids on ‘Golden Delicious’ apples treated
with different adjuvants. Mills River, NC. 2011.

| | Total aphids on the most-infested leaf on each of 10 shoots | | | | | |
|---|---|---|---|---|---|---|
| Treatment | 18-May | 25-May | 2-Jun | 8-Jun | 17-Jun | Season total |
| Alkoxylated Alkanol Adjuvant (0.25%) + | 13.8a | 14.3a | 1.0a | 0.3a | 0.0a | 29.3a |
| Insecticide | | | | | | |
| Alkoxylated Alkanol Adjuvant (0.5%) + | 3.0a | 0.3a | 0.5a | 0.0a | 0.0a | 3.8a |
| Insecticide | | | | | | |
| Alkoxylated Alkanol Adjuvant (0.5%), | 20.8a | 2.0a | 0.0a | 0.0a | 0.0a | 22.8a |
| No Insecticide | | | | | | |
| Kinetic ® (0.25%) + Insecticide | 0.5a | 0.0a | 0.0a | 0.0a | 0.0a | 0.5a |
| Control | 0.0a | 0.8a | 0.3a | 0.5a | 1.0a | 2.5a |

Means in the same column followed by the same letter are not significantly different by LSD (p = 0.05).

TABLE 10
Damage at harvest (8-Sep) on ‘Golden Delicious’ apples
treated with different adjuvants. Mills River, NC. 2011.

| | % Damage | | | | | | |
|---|---|---|---|---|---|---|---|
| | | | Plum | | Comstock | Apple | % Clean |
| Treatment | Lep stings | Lep entries¹ | curculio | Plant bug | mealy bug | maggot | fruit |
| Alkoxylated Alkanol Adjuvant | 0.5a | 2.0a | 0.5a | 2.0a | 0.5a | 0.0a | 94.5a |
| (0.25%) + Insecticide | | | | | | | |
| Alkoxylated Alkanol Adjuvant | 2.0a | 4.0ab | 16.0a | 0.5a | 0.5a | 0.5a | 67.0a |
| (0.5%) + Insecticide | | | | | | | |
| Alkoxylated Alkanol Adjuvant | 1.0a | 13.0bc | 4.0a | 2.0a | 14.0b | 3.0a | 61.0a |
| (0.5%), No Insecticide | | | | | | | |
| Kinetic ® (0.25%) + Insecticide | 2.5a | 5.5ab | 7.5a | 5.0a | 0.0a | 1.0a | 78.5a |
| Control | 1.0a | 20.5c | 10.0a | 4.0a | 2.0a | 1.5a | 57.5a |

Means in the same column followed by the same letter are not significantly different by LSD (p = 0.05).
¹Data were transformed by sqrt(x + 1). Data presented are back transformations.

TABLE 11

| | | GreenLeaf | | |
|---|---|---|---|---|
| | | FullCote 0.25% | Kinetic 0.25% | Statistically |
| | | in pesticide | in pesticide | Significant |
| Plant | Pest | blend | blend | Difference? |
| Cabbage | Thrips | 42.3 Thrips/ | 53.0 Thrips/ | No |
| | | 5 plants | plant | |
| | Flea | 0.8 Beetles/ | 0.6 Beetles/ | No |
| | Beetles | 5 plants | plant | |
| Tomato | Adult | 72.6 Thrips/ | 70.0 Thrips/ | No |
| | Thrips | 10 Flowers | 10 Flowers | |
| | Potato Aphid | Equal Effect | Equal Effect | No |
| Apples | European | 3.0 cumulative | 6.0 cumulative | No |
| | Red Mite | mite days | mite days | |
| | Potato | 9.5 Total | 17.3 Total | No |
| | Leafhoppers | | | |

A number of variations and modifications of the disclosure can be used. One of more embodiments of the disclosure can used separately and in combination. That is, any embodiment alone can be used and all combinations and permutations thereof can be used. It would be possible to provide for some features of the disclosure without providing others.

The present disclosure, in various embodiments, configurations, or aspects, includes components, methods, processes, systems and/or apparatus substantially as depicted and described herein, including various embodiments, configurations, aspects, sub-combinations, and subsets thereof. Those of skill in the art will understand how to make and use the various embodiments, configurations, or aspects after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that any claim and/or combination of claims require more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment.

Moreover, though the description of the disclosure has included descriptions of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An aqueous slurry composition, comprising:
an active agent; and
an alkoxylated alkanol having from about 1 to about 30 moles of alkoxylate, the alkanol having the following structure: 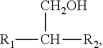
where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof.

2. The composition of claim 1, wherein the active agent is one of a lubricant, anti-static agent, scouring agent, herbicide, insecticide, fungicide, foliar nutrient, plant growth regulator or a mixture thereof, wherein the alkoxylated alkanol adjuvant has from about 1 to about 5 moles of the alkoxylate, wherein the total number of carbon atoms of R₁ and R₂ are from about 1 to about 18, wherein R₂ is CH₃ and R₁ contains at least two branch sites.

3. The composition of claim 1, wherein the alkoxylated alkanol adjuvant comprises from about 1 to about 3 moles of the alkoxylate.

4. The composition of claim 1, wherein the alkoxylated alkanol adjuvant comprises about 2 moles of the alkoxylate.

5. The composition of claim 1, wherein the alkoxylate comprises ethylene oxide.

6. The composition of claim 1, wherein the alkoxylate comprises propylene oxide.

7. The composition of claim 1, wherein the alkoxylate comprises a mixture of ethylene oxide and propylene oxide.

8. The composition of claim 1, wherein the composition comprises no more than about 1 wt % of the alkoxylated alkanol adjuvant.

9. The composition of claim 1, wherein the composition comprises from about 0.25 wt % to about 0.5 wt % of the alkoxylated alkanol adjuvant.

10. The composition of claim 1, wherein the alkanol comprises one of a C₁₂, C₁₃, C₁₄, C₁₅, C₁₆, C₁₇, C₁₈ alkanol or mixture thereof.

11. The composition of claim 1, wherein the alkoxylate alkanol adjuvant comprises a C₁₂-C₁₈ alkanol having about 2 moles ethylene oxide.

12. The composition of claim 1, wherein the adjuvant comprises isostearyl alcohol having two moles ethylene oxide.

13. A method for treating a plant, comprising:
applying to a least a portion of a surface of a plant an effective amount of a slurry composition having:
an active agent; and
an alkoxylated alkanol adjuvant having from about 1 to about 30 moles of alkoxylate, the alkanol having the following structure: 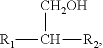
where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof.

14. The method of claim 13, wherein the active agent is an herbicide, insecticide, fungicide, foliar nutrient, and plant growth regulator, or mixture thereof, wherein the alkoxylated alkanol adjuvant has from about 1 to about 5 moles of the alkoxylate, wherein the total number of carbon atoms of R₁ and R₂ are from about 1 to about 18, wherein R₂ is CH₃ and R₁ contains at least two branch sites.

15. The method of claim 13, wherein the applying comprises a process selected from the group consisting of spraying, painting, and dipping.

16. The method of claim 13, wherein the plant is selected from the group consisting of fruits, vegetables, grains, legumes, tress, scrubs, flowers, grasses, roots, landscape plants, ornamental plants, and crop plants.

17. The method of claim 13, wherein the alkoxylated alkanol adjuvant comprises from about 1 to about 3 moles of the alkoxylate.

18. The method of claim 13, wherein the alkoxylated alkanol adjuvant comprises about 2 moles of the alkoxylate.

19. The method of claim 13, wherein the alkoxylate comprises ethylene oxide.

20. The method of claim 13, wherein the alkoxylate comprises propylene oxide.

21. The method of claim 13, wherein the alkoxylate comprises a mixture of ethylene oxide and propylene oxide.

22. The method of claim 13, wherein the composition comprises no more than about 1 wt % of the alkoxylated alkanol adjuvant.

23. The method of claim 13, wherein the composition comprises from about 0.25 wt % to about 0.5 wt % of the alkoxylated alkanol adjuvant.

24. The method of claim 13, wherein the alkanol comprises one of a C₁₂, C₁₃, C₁₄, C₁₅, C₁₆, C₁₇, C₁₈ alkanol or mixture thereof.

25. The method of claim 13, wherein the alkoxylated alkanol adjuvant comprises a C₁₂-C₁₈ alkanol having about 2 moles ethylene oxide.

26. The method of claim 13, wherein the alkoxylated alkanol adjuvant comprises isostearyl alcohol having two moles ethylene oxide.

27. The method of claim 13, wherein the treating of the plant comprises protecting the plant from pest infestation.

28. The method of claim 13, wherein the applying of the slurry composition further comprises:
applying the slurry to one of a first and second surface portion of the plant, wherein, thereafter the applying of the slurry, the slurry spreads and/or wets the other of the first and second surface portions.

29. A coated non-fluid substrate, wherein at least a portion of the non-fluid substrate surface is coated with an aqueous composition containing a branched, alkoxylated alkanol adjuvant having from about 1 to about 30 moles of alkoxylate, the alkanol having the following structure: 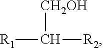
where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof.

30. The coated surface of claim 29, wherein the non-fluid substrate comprises one of earth, a flooring material, a table, a counter top, a fiber, a fiber containing material, a plant or plant product, a plant by-product, an animal, an animal by-product, a polymeric material, a natural occurring material, a metal or metal containing material, glass, wood, a composite material, a ceramic, or combinations and mixtures thereof, wherein the alkoxylated alkanol adjuvant has from about 1 to about 5 moles of the alkoxylate, wherein the total number of carbon atoms of R₁ and R₂ are from about 1 to about 18, wherein R₂ is CH₃ and R₁ contains at least two branch sites.

31. A method for treating a non-fluid substrate, comprising:
contacting to a portion of a non-fluid substrate surface with an aqueous composition containing a branched, alkoxylated alkanol adjuvant having about 1 to about 30 moles of alkoxylate, the alkanol having the following structure: 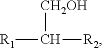
where the total number of carbon atoms of R₁ and R₂ are from about 3 to about 36, R₂ can be hydrogen or C₁ to C₁₈, and R₂ may or may not be branched, and wherein the alkoxylate is one of ethylene oxide, propylene, or mixture thereof; and
wherein, thereafter the contacting of the slurry with the non-fluid substrate, the slurry spreads and/or wets a substantial portion of the non-fluid substrate not contacted with the aqueous solution.

32. The coated surface of claim 29, wherein the non-fluid substrate comprises one of earth, a flooring material, a table or counter top, a fiber or fiber containing material, a plant or plant product, a mold, or combination thereof, wherein the alkoxylated alkanol adjuvant has from about 1 to about 5 moles of the alkoxylate, wherein the total number of carbon atoms of R₁ and R₂ are from about 1 to about 18, wherein R₂ is CH₃ and R₁ contains at least two branch sites.
